(19) United States
(12) Patent Application Publication
(10) Pub. No.: US 2026/0283841 A1
(43) Pub. Date: Sep. 24, 2026

(54) MOBILE PALMAR COOLING TECHNOLOGY

(71) Applicant: ARTERIA TECHNOLOGY, INC., Boca Raton, FL (US)

(72) Inventors: Bria Romero, Boca Raton, FL (US); Josh Goforth, Boca Raton, FL (US); Dylan Ueyama-Burke, Boca Raton, FL (US); Tiffany Vuong, Boca Raton, FL (US); Aman Eujayl, Boca Raton, FL (US)

(21) Appl. No.: 19/475,377

(22) PCT Filed: Apr. 15, 2024
(86) PCT No.: PCT/US2024/024627

§ 371 Date: Oct. 14, 2025

Related U.S. Application Data
(60) Provisional application No. 63/459,219, filed on Apr. 13, 2023.

(51) Int. Cl. A61F 7/00

(52) U.S. Cl. CPC A61F 7/007; A61F 2007/0036; A61F 2007/0056; A61F 2007/0075; A61F 2007/0078; A61F 2007/0093

(57) ABSTRACT

A portable cooling unit includes a conductive plate having a first side and a second side. The portable cooling unit includes a heat sink having an upper side, a lower side, and a body thermally connecting the upper side and the lower side. The portable cooling unit includes a fan having an air outlet side and an air inlet side. The portable cooling unit optionally includes one or more heat pipes thermally connecting the conductive plate to the heat sink. The portable cooling unit optionally includes a Peltier module for dissipating heat from the conductive plate and/or the heat pipes.

### CROSS-REFERENCE TO RELATED APPLICATIONS

This application claims benefit of and priority to U.S. Provisional Application No. 63/459,219 filed on Apr. 13, 2023, and titled “Mobile Palmar Cooling Technology,” the contents of which are incorporated herein their entirety.

### FIELD

The present disclosure relates to portable and/or wearable devices to control core body temperature of a user.

### BACKGROUND

Elevated core body temperature can be induced by exercise, stress, or medical conditions that affect thermoregulation. As a result of an increase in core body temperature, pyruvate kinase, an enzyme involved in the creation of energy-providing adenosine triphosphate (ATP) molecules, becomes denatured. Additionally, the brain activates fatigue signals, and heat exhaustion also sets in as a result of the elevation in core body temperature. If the heat exhaustion is not addressed in a timely manner, it can even result in severe consequences, such as syncope and heat strokes.

### SUMMARY

Disclosed in this specification are approaches for addressing various of the problems and shortcomings of the state of the art, as identified above. More particularly, disclosed herein are portable cooling units that can be worn by a user to reduce core body temperature and combat heat exhaustion.

According to a first aspect of the present disclosure, there is provided a portable cooling unit comprising a conductive plate having a first side and a second side, a heat sink having an upper side, a lower side, and a body thermally connecting the upper side and the lower side, one or more heat pipes thermally connecting the conductive plate to the heat sink, a fan having an air outlet side and an air inlet side, and a power source. The upper side of the heat sink is positioned facing the second side of the conductive plate, the fan is connected to the heat sink such that the air outlet side is positioned facing the lower side of the heat sink, and the power source is connected to the fan via at least one connecting cable.

According to a second aspect of the present disclosure, there is provided a portable cooling unit comprising a conductive plate having a first side and a second side, the first side of the conductive plate configured to interface with a palm of a user, the first side of the conductive plate configured to be secured with the palm of the user via at least one connecting strap. The portable cooling unit comprises a heat sink having an upper side, a lower side, and a body thermally connecting the upper side and the lower side, one or more heat pipes thermally connecting the conductive plate to the heat sink, a fan having an air outlet side and an air inlet side, and a power source. The upper side of the heat sink is positioned facing the second side of the conductive plate. The heat sink comprises a plurality of fins disposed on the body to increase a surface area of the heat sink for heat dissipation. The fan is connected to the heat sink such that the air outlet side is positioned facing the lower side of the heat sink. The power source is connected to the fan via at least one connecting cable. The power source is configured to be secured to a wrist of the user via at least one wrist strap.

According to a third aspect of the present disclosure, there is provided a portable cooling unit comprising a conductive plate having a first side and a second side, a heat sink having an upper side, a lower side, and a body thermally connecting the upper side and the lower side, a power source. The portable cooling unit comprising a Peltier module electrically connected to the power source via the at least one connecting cable. The upper side of the heat sink is positioned facing the second side of the conductive plate, and the heat sink comprises a plurality of fins disposed on the body to increase a surface area of the heat sink for heat dissipation. The Peltier module comprises a heat absorbing end thermally connected to the second side of the conductive plate and configured to absorb heat from the conductive plate, a heat releasing end thermally connected to the upper side of the heat sink and configured release the absorbed heat from the conductive plate into the heat sink, and a plurality of semiconductor elements electrically connected to the power source, the plurality of semiconductor elements thermally connecting the heat absorbing end and the heat releasing end.

According to a fourth aspect of the present disclosure, there is provided a portable cooling unit comprising a conductive plate having a first side and a second side, a heat sink having an upper side, a lower side, and a body thermally connecting the upper side and the lower side, one or more heat pipes thermally connecting the conductive plate to the heat sink, a fan having an air outlet side and an air inlet side, a power source. The portable cooling unit comprising a Peltier module electrically connected to the power source via the at least one connecting cable. The upper side of the heat sink is positioned facing the second side of the conductive plate, the fan is connected to the heat sink such that the air outlet side is positioned facing the lower side of the heat sink, and the power source is connected to the fan via at least one connecting cable. The heat sink comprises a plurality of fins disposed on the body to increase a surface area of the heat sink for heat dissipation. The Peltier module comprises a heat absorbing end thermally connected to the second side of the conductive plate and configured to absorb heat from the conductive plate, a heat releasing end thermally connected to the upper side of the heat sink and configured release the absorbed heat from the conductive plate into the heat sink, and a plurality of semiconductor elements electrically connected to the power source, the plurality of semiconductor elements thermally connecting the heat absorbing end and the heat releasing end.

According to a fifth aspect of the present disclosure, there is provided a portable cooling unit comprising a conductive plate having a first side and a second side, a heat sink having an upper side, a lower side, and a body thermally connecting the upper side and the lower side, the upper side of the heat sink positioned facing the second side of the conductive plate, the heat sink comprising a plurality of fins disposed on the body, the plurality of fins being configured to increase a surface area of the heat sink for heat dissipation. The portable cooling unit includes a power source; and a Peltier module electrically connected to the power source via at least one connecting cable. The Peltier module comprises a heat absorbing end thermally connected to the second side of the conductive plate and configured to absorb heat from the conductive plate; a heat releasing end thermally connected to the upper side of the heat sink and configured release the absorbed heat from the conductive plate into the heat sink; and a plurality of semiconductor elements electrically connected to the power source, the plurality of semiconductor elements thermally connecting the heat absorbing end and the heat releasing end.

According to a sixth aspect of the present disclosure, there is provided a portable cooling unit comprising a conductive plate having a first side and a second side; a heat sink having an upper side, a lower side, and a body thermally connecting the upper side and the lower side, the upper side of the heat sink positioned facing the second side of the conductive plate, and the heat sink comprising a plurality of fins disposed on the body, the plurality of fins being configured to increase a surface area of the heat sink for heat dissipation. The portable cooling unit comprises a fan having an air outlet side and an air inlet side, wherein the fan is connected to the heat sink such that the air outlet side is positioned to face the lower side of the heat sink, The portable cooling unit comprises a power source connected to the fan via at least one connecting cable. The portable cooling unit comprises one or more heat pipes and a Peltier module electrically connected to the power source via at least one connecting cable. The Peltier Module comprises a heat absorbing end thermally connected to the one or more heat pipes and configured to absorb heat from the one or more heat pipes; a heat releasing end thermally connected to the upper side of the heat sink and configured release the absorbed heat from the one or more heat pipes into the heat sink; and a plurality of semiconductor elements electrically connected to the power source, the plurality of semiconductor elements thermally connecting the heat absorbing end and the heat releasing end.

### BRIEF DESCRIPTION OF THE DRAWINGS

For clarity and understanding, some implementations of the technologies are described in more detail in the appended drawings. These drawings should not, however, be considered as limiting the scope of the inventions and the inventions include any number of other equally useful implementations.

FIG. 1 shows a schematic diagram of an exemplary portable cooling unit as described in this specification.

FIG. 2 shows a schematic diagram of an exemplary implementation of a conductive plate of a the portable cooling unit as described in this specification.

FIG. 3 shows a cross-sectional schematic diagram of an exemplary implementation of a heat pipe of a portable cooling unit as described in this specification.

FIG. 4 shows a perspective view of an exemplary implementation of a heat sink of a portable cooling unit as described in this specification.

FIG. 5 shows a schematic diagram of an exemplary implementation of a Peltier module of a portable cooling unit as described in this specification.

FIG. 6 shows a schematic diagram of an exemplary implementation of a fan according of a portable cooling unit as described in this specification.

FIG. 7 shows a schematic diagram of an exemplary implementation of a liquid cooling system of a portable cooling unit as described in this specification.

FIG. 8 shows an exemplary schematic of a temperature control system of a portable cooling unit as described in this specification.

FIG. 9 shows flow diagram of an exemplary method of operation of the temperature control system.

FIG. 10 shows a diagram of an exemplary portable cooling unit as described in this specification disposed on the body of a user.

FIG. 11 shows a diagram of an exemplary handpiece of a portable cooling unit as described in this specification.

FIG. 12 shows a perspective schematic view of an exemplary portable cooling unit.

FIG. 13A-13C show schematic diagrams of exemplary portable cooling units as described in this specification.

FIG. 14A-14C shows schematic diagrams of exemplary portable cooling unit as described in this specification.

### DETAILED DESCRIPTION

The concept of palmar cooling can be utilized to reduce core body temperature and combat heat exhaustion. In the palms of human hands, there is a network of veins that specialize in heat transfer called arteriovenous anastomoses (AVAs). In response to increase in core body temperature, AVAs vasodilate, and blood flows rapidly to the periphery of the body (e.g., the palms). Through circulation, blood cools and in turn reduces the core body temperature. The concept of palmar cooling has been shown to improve performance and endurance in high-activity individuals such as athletes.

A mobile/portable palmar cooling unit can be used to utilize the concept of palmar cooling to reduce the core body temperature as discussed above. However, existing palmar cooling technologies generally tend to be bulky, inefficient, expensive, difficult to use, and have a poor battery life. There remains an unresolved and unfulfilled need in the art for a mobile palmar cooling device that is inexpensive, has greater mobility/portability, efficient, easy to use, and has adequate battery life.

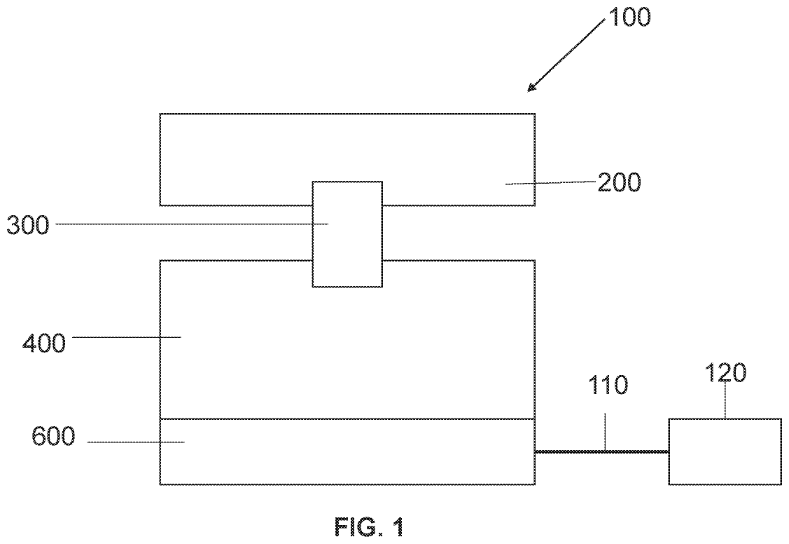

Referring to the drawings, FIG. 1 shows a schematic diagram of an exemplary portable cooling unit 100. The portable cooling unit 100 is designed to interact with a palm of a user and reduce core temperature via the concept of palmar cooling. FIG. 1 also presents structural components of the example portable cooling unit 100. These include a conductive plate 200, a heat sink 400, a heat pipe 300 thermally connecting the conductive plate 200 and the heat sink 400, a power source 120, and a fan 600. As shown in FIG. 1, the fan 600 is coupled to the heat sink 400 and electrically connected to the power source 120 via a connecting cable 110.

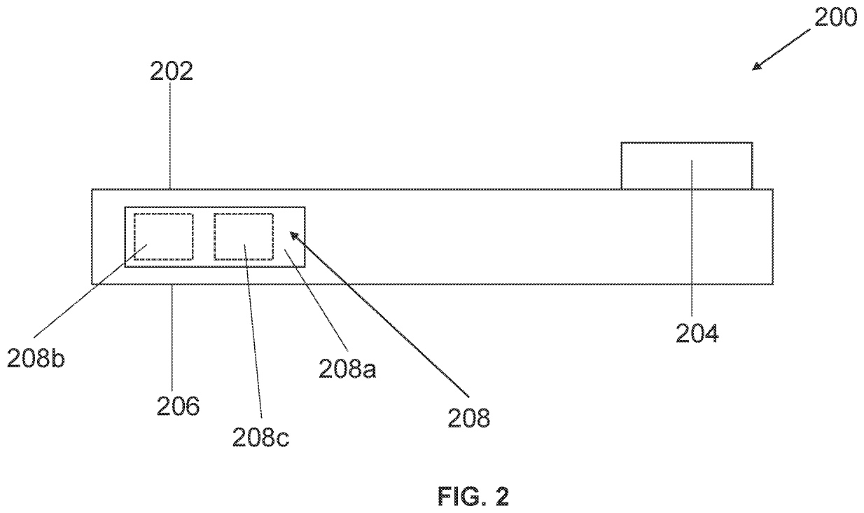

FIG. 2, shows a schematic diagram of an exemplary implementation of the conductive plate 200 of the portable cooling unit 100. The conductive plate 200 (as shown in FIG. 2) includes a first side 202 for interfacing with the palm of the user, a temperature probe 204 disposed on a surface of the first side 202, a second side 206 thermally connected to the first side 202, and a user interface 208 for interaction with the user. In some implementations, the conductive plate 200 can have different shapes and/or sizes according to a size of the palm of the user. An example conductive plate 200 is substantially flat, e.g., because the palm needs to be in a relaxed state, as opposed to a fisted or fully extended shape, e.g., to prevent or reduce mechanical constriction of blood vessels (e.g., of the AVAs). A conductive plate 200 can have any shape (e.g., an ergonomic shaper, e.g., curved, tubular, etc.) to maximize surface contact with the skin, e.g., to maximize heat transfer while staying above a predetermined temperature threshold (e.g., 13° C.). In some implementations of the conductive plate 200, the conductive plate 200 is made from a conductive metal or metal alloy, such as copper, copper-alloys, stainless steel, aluminum, or combinations thereof.

In the implementation of the conductive plate 200 presented in FIG. 2, heat from the palm of the user is absorbed via the first side 202 of the conductive plate 200, and the absorbed heat is dissipated via the second side 206 of the conductive plate 200. In some implementations of the conductive plate 200, the first side 202 of the conductive plate 200 can include an insulation layer (not shown), e.g., to prevent heat escaping via the first side 202 of the conductive plate 200, or to prevent damage to skin (“ice burn”) due to excessive cooling, e.g., due to equipment malfunction. Preventing the heat from escaping via the first side 202 of the conductive plate 200 can prevent the heat from reverting to the palm of the user and reducing the efficiency of the portable cooling unit 100 in reducing the core body temperature of the user. In some implementations of the conductive plate 200, the insulation layer is made of an insulative material, e.g., thermoplastic polyurethane (TPU) or cloth fabric.

Referring to the implementation of the conductive plate 200 presented in FIG. 2, the user interface 208 disposed on the conductive plate 200 includes a graphic user interface (GUI) 208a, a processor 208b coupled to the GUI 208a, and a memory 208c coupled to the processor 208b. In some implementations of the conductive plate 200, the GUI 208a can be a liquid clear display (LCD) panel that displays the temperature of the surface of first side 202 of the conductive plate 200 (e.g., via the temperature probe 204 electrically connected to processor 208b). In some implementations of the conductive plate 200, the GUI 208a can be an LCD panel that displays environmental conditions (e.g., ambient temperature) received via an external probe (not shown in FIG. 2) measuring environmental conditions. The GUI can include an indicator of unacceptable environmental conditions (e.g., a thermometer), including conditions of one or mor components of the portable cooling unit 100, e.g., the heat sink 400 and/or the cooling charger described below. In other implementations of the conductive plate 200, the GUI 208a can be an LCD touch-screen panel for user interaction (e.g., for controlling the temperature of the surface of the first side 202 of the conductive plate 200) to control the portable cooling unit 100. Examples of a system and method of controlling the temperature of the surface of the first side 202 of the conductive plate 200 are presented below.

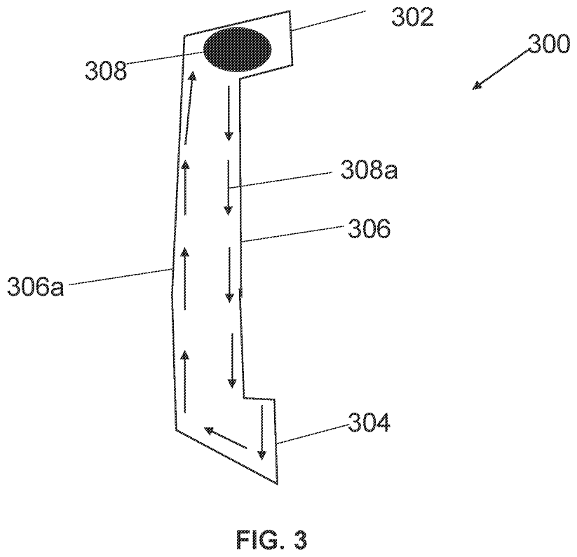

FIG. 3, shows a cross-sectional schematic diagram of an exemplary implementation of the heat pipe 300 for an example portable cooling unit 100. The implementation of the heat pipe 300 shown in FIG. 3 includes a heat interface 302, a cold interface 304, a tunnel 306 thermally connecting the heat interface 302 and the cold interface 304, and a volatile liquid 308 contained within the tunnel 306. In the implementation of the heat pipe 300 presented in FIG. 3, the heat interface 302 of the heat pipe 300 thermally interacts with the conductive plate 200 (e.g., on the second side 206 of conductive plate 206) to receive the heat absorbed from the palm of the user. In an implementation, a section of heat pipe 300 is at least partially embedded in conductive plate 200. The heat received by the heat pipe 300, e.g., at heat interface 302, is transported via the tunnel 306 by the volatile liquid 308 present in the tunnel 306. When the volatile liquid 308 receives heat, it undergoes a change of physical state from the volatile liquid 308 to a vapor 308a and travels from the heat interface 302 to the cold interface 304, transporting the heat away from the conductive plate 200. In some implementations of the heat pipe 300, the volatile liquid 308 can be a liquid with a low-boiling point, such as a refrigerant. In some implementations of the heat pipe 300, the volatile liquid 308 can include water. In some implementations of the heat pipe 300, the heat pipe 300 can be made of a heat conductive material, such as copper, aluminum, steel, or a combination thereof. In an implementation, heat pipe 300 is substantially tubular. In an implementation, heat pipe 300 has a serpentine configuration e.g., to maximize contact surface between conductive plate 200/second side 206 and heat pipe 300. In an implementation, heat pipe 300 has a substantially flat heat interface 302 and/or a substantially flat cold interface 304. In some implementations of the heat pipe 300, the heat pipe 300 can be shaped to be thin and flattened into the same shape as the conductive plate 200, e.g., to maximize contact surface between conductive plate 200/second side 206 and heat pipe 300.

In the implementation of the heat pipe 300 presented in FIG. 3, the vapor 308a is transported to cold interface 304 where it undergoes a change of physical state from the vapor 308a to the volatile liquid 308, before being transported back to the heat interface 302 via the tunnel 306 by capillary action. In some implementations, the tunnel 306 can include an interior surface 306a configure to facilitate capillary action. In some implementations, the interior surface 306a of the tunnel 306 can include a wick connecting the cold interface 304 and heat interface 302, such that the condensed volatile liquid 308 can be transported from the cold interface 304 to the heat interface 302 via capillary action.

In the implementation of the heat pipe 300 presented in FIG. 3, the cold interface 304 of the heat pipe 300 is thermally connected to a heat sink 400 such that the heat released during the condensation of the vapor 308a is released into the heat sink 400 for heat dissipation.

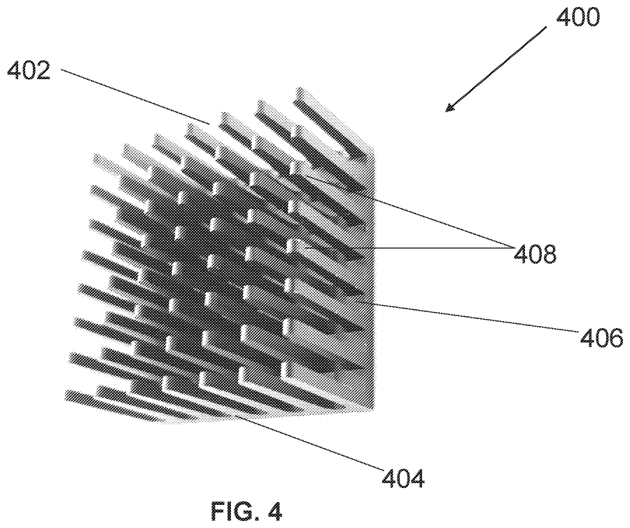

FIG. 4 shows a perspective view of an exemplary implementation of a heat sink 400 for a portable cooling unit 100. In the implementation of the heat sink 400 shown in FIG. 4, the heat sink 400 includes an upper side 402, a lower side 404, a body 406 thermally connecting the upper side 402 and the lower side 404, and a plurality of fins 408 disposed on the body 406. In one implementation of the heat sink 400 presented in FIG. 4, the upper side 402 of the heat sink 400 is configured to receive heat from the cold interface 304 of the heat pipe 300. In the same implementation of the heat sink 400, the heat received by the upper side 402 of the heat sink 400 is transported via the body 406 to be dissipated via the lower side 404 of the heat sink 400 and/or the plurality of fins 408. The plurality of fins 408 increase the surface area of the heat sink 400 for the heat to be dissipated into the ambient air. In some implementations of the heat sink 400, the heat sink 400 is made out of a conductive material, e.g., copper, aluminum, or alloys of aluminum, to transport the heat from the upper side 402 of the heat sink 400 to the lower side 404 of the heat sink 400 via the process of conduction.

In some implementations of the heat sink 400, the heat sink 400 can dissipate the heat via the plurality of fins 408 and the lower side 404 of the heat sink 400 via radiation, conduction, convection, or a combination of at least two of the processes of heat dissipation. In some implementations of the heat sink 400, the capacity of the heat sink 400 to dissipate heat can be increased by thermally coupling the heat sink 400 with a cooling element to cool the body 406 of the heat sink 400. In some implementations of the heat sink 400, the cooling element can include a coolant or refrigerant, e.g., a phase-change material including, e.g., water, a hydrocarbon, a fluorocarbon, a salt hydrate, or a combination thereof, e.g., a material that can be cooled to different temperatures (i.e., frozen to ice) based on the need for increasing the capacity of the heat sink 400. In some implementations of the heat sink 400, the cooling element can be an electrical cooling element disposed on the heat sink 400 and configured to increase the capacity of the heat sink 400 for heat dissipation (e.g., a fan or a thermoelectric cooling element). In some implementations, heat sink 400 can be or can include a (separate, e.g., removable) cooling module (“cooling charger”) including a container with a phase change material, e.g., a hydrocarbon, a chlorofluorocarbon, or a fluorocarbon (e.g., perfluoropentane). This cooling charger can be cooled in a freezer or refrigerator prior to use on the portable cooling unit 100.

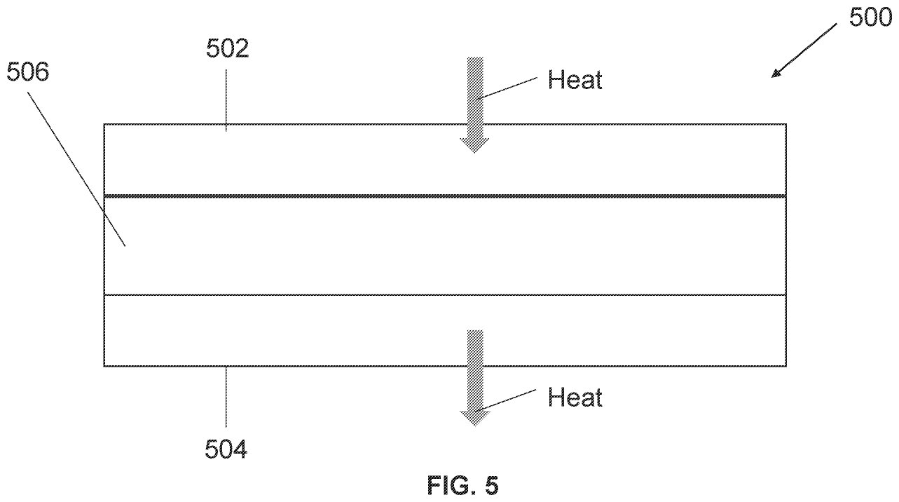

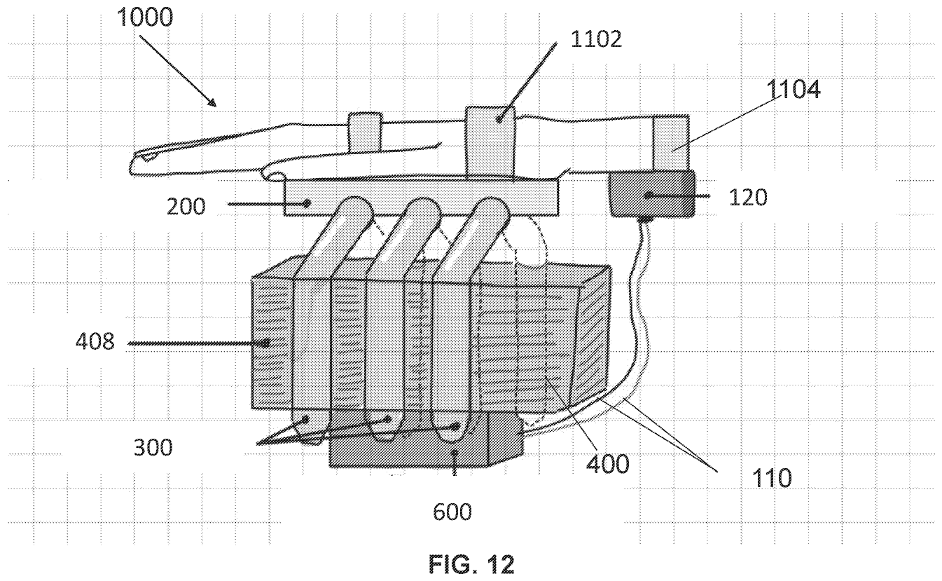

FIG. 5 shows a schematic diagram of an exemplary implementation of a Peltier module 500 (an electrical/thermoelectric cooling element) for a portable cooling unit 100. In the implementation of the Peltier module shown in FIG. 5, the Peltier module 500 includes a heat absorbing end 502, a heat releasing end 504, and a plurality of semiconductor elements 506 connected to the power source 120 and thermally connecting the heat absorbing end 502 and the heat releasing end 504. In some implementations of the Peltier module 500, the heat absorbing end 502 of the Peltier module 500 is thermally connected to the second side 206 of the conductive plate 200 to receive the heat from the conductive plate 200. In some implementations of the Peltier module 500, the heat absorbing end 502 end of the Peltier module 500 can be thermally connected to the cold interface 304 of the heat pipe 300 and configured to receive/dissipate heat from the heat pipe 300. In some implementations of the heat sink 400, the Peltier module 500 can be disposed on a surface of heat sink 400 to increase the heat dissipation capacity of the heat sink 400. In an implementation of the Peltier module 500, the heat releasing end 504 of the Peltier module 500 can be disposed on the upper side 402 of the heat sink 400 to release the received heat into the heat sink 400 for heat dissipation (e.g., as shown in FIG. 12B below). In an implementation of the Peltier module 500, the heat absorbing end 502 of the Peltier module 500 can be disposed on the heat sink 400 to absorb heat from the heat sink 400 for heat dissipation.

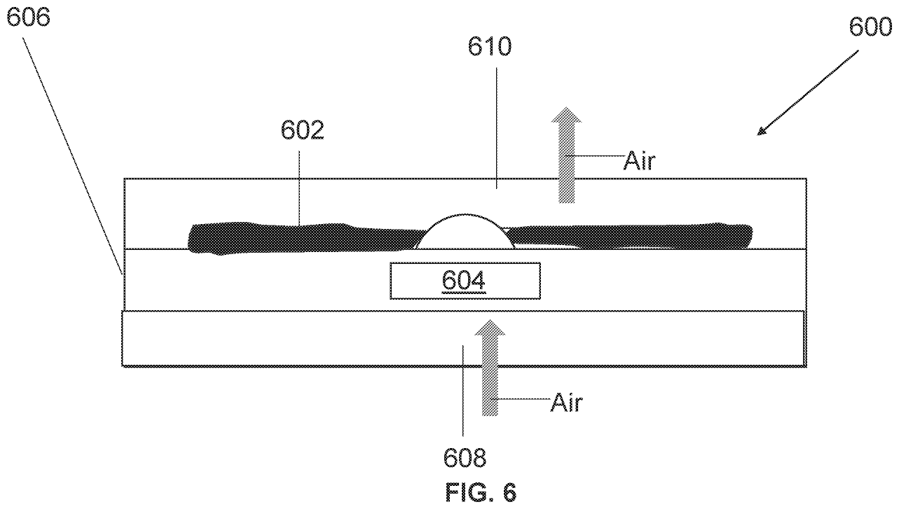

In some implementations, to further increase the capacity of the heat sink 400 to dissipate heat, a fan 600 can be connected to heat sink 400 to cool the heat sink 400. FIG. 6 shows a schematic diagram of an exemplary implementation of a fan 600 for a portable cooling unit 100. The implementation of the fan 600 shown in FIG. 6 includes a motor 604, fan blades 602 actuated by the motor 604, a housing 606, an air inlet 608 and an air outlet 610. In some implementations of the portable cooling device 100, the fan 600 can be positioned in a way that the air outlet 610 of the fan 600 is facing the lower side 404 of the heat sink 400 to cool down the heat sink 400 when the fan blades 602 are actuated. In an implementation of the fan presented in FIG. 6, the fan 600 can be powered by the power source 120 connected to fan 600 via a connecting cable 110. In some implementations of the fan 600, the fan 600 can further include an in-built power source in housing 606 to power the motor 604 and actuate the fan blades 602 without the need for connection with a power source 120. In some implementations of the fan 600, the fan 600 can have a bladeless design. In other implementations of the fan 600, the actuation of the fan blades 602 and the speed of the actuation of the fan blades 602 can be controlled via a user interface, e.g., the user interface 208 on the conductive plate 200.

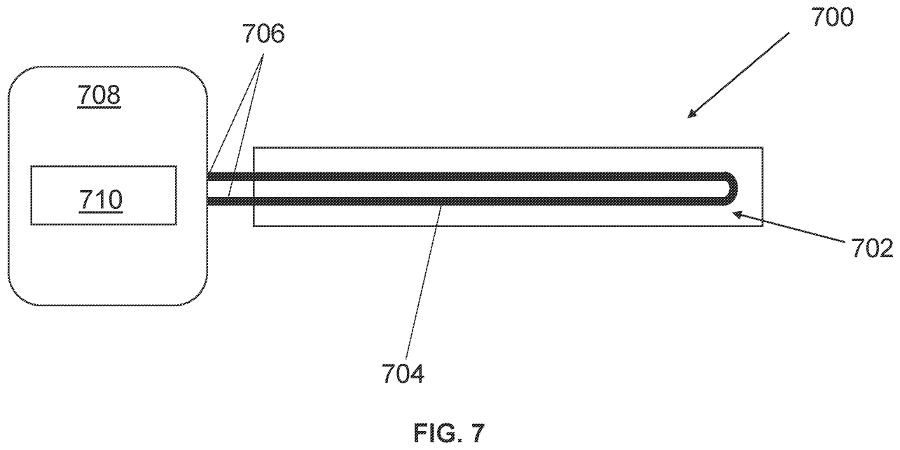

FIG. 7, shows a schematic diagram of an exemplary implementation of a liquid cooling system 700 for a portable cooling unit 100. The liquid cooling system 700 can be used to increase the efficiency of the portable cooling unit 100 by increase a rate of heat transfer through the conductive plate 200. In an implementation of the liquid cooling system 700 shown in FIG. 7, the liquid cooling system 700 includes an internal fluid chamber 702 containing conductive tubing 704, a pump 708 connected to the conductive tubing 704 via fluid ports 706, where the pump 708 is electrically connected to the power source 120, e.g., via a connecting cable 110. Pump 708 is fluidically connected to a reservoir 710. In an implementation of the liquid cooling system 700 shown in FIG. 7, the pump 708 includes a reservoir 710 containing a liquid coolant/refrigerant for flowing through the conductive tubing 704. In an implementation of the liquid cooling system 700 shown in FIG. 7, the internal fluid chamber 702 is disposed on the second side 206 of the conductive plate 200. The liquid coolant flowing through the conductive tubing 704 can be configured to receive the heat from the conductive plate 200 and actively cool the conductive plate 200. In some implementations of the liquid cooling system 700, the conductive tubing 704 can be made from a thermally conductive material, e.g., copper. In some implementations of the liquid cooling system 700, the conductive tubing 704 can be serpentine-shaped to increase surface area for absorbing the heat from the conductive plate 200. In some implementations of the liquid cooling system 700, the liquid coolant can be a heat sensitive phase-change liquid including, e.g., water, a hydrocarbon, a chlorofluorocarbon, a fluorocarbon (e.g., perfluoropentane), a salt hydrate, or a combination thereof. In other implementations of the liquid cooling system 700, the liquid coolant can include water.

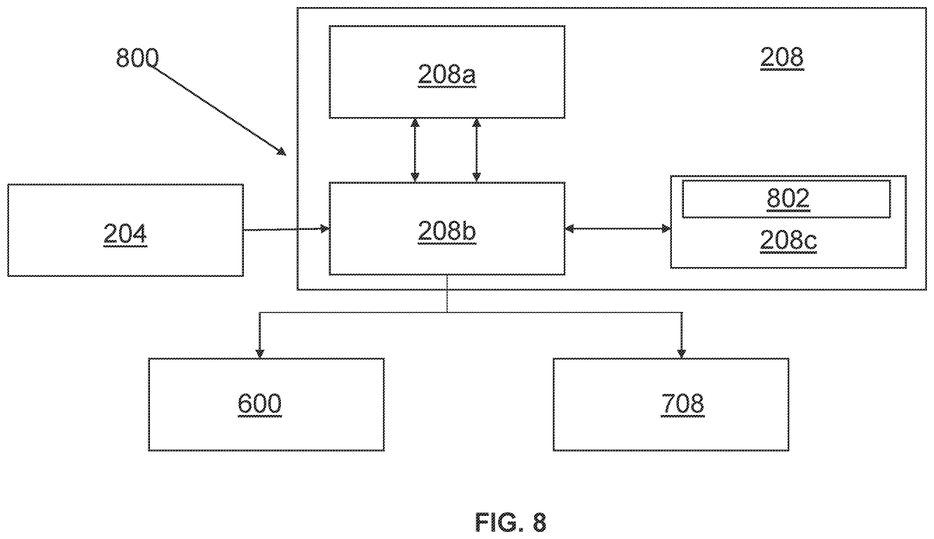

FIG. 8 shows an exemplary schematic of a temperature control system 800 for a portable cooling unit 100. In an implementation of the temperature control system 800 as shown in FIG. 8, the temperature control system 800 includes or is electronically connected to the user interface 208 connected and/or to the temperature probe 204, the fan 600, and the pump 708. In the example schematic presented in FIG. 8, the user interface 208 includes a GUI 208a, a processor 208b in communication with the GUI 208a, and a memory 208c coupled to the processor 208b and including instructions 802 for the processor 208b to carry out a set of operations of the temperature control system 800. In the implementation of the temperature control system 800 shown in FIG. 8, the user interface 208 is also configured to receive temperature values of the surface of the first side 202 of conductive plate 200 from the temperature probe 204. In some implementations of the temperature control system 800, the temperature control system 800 can be connected to the Peltier module 500 configured to dissipate heat from the conductive plate 200 and/or the heat pipes 300.

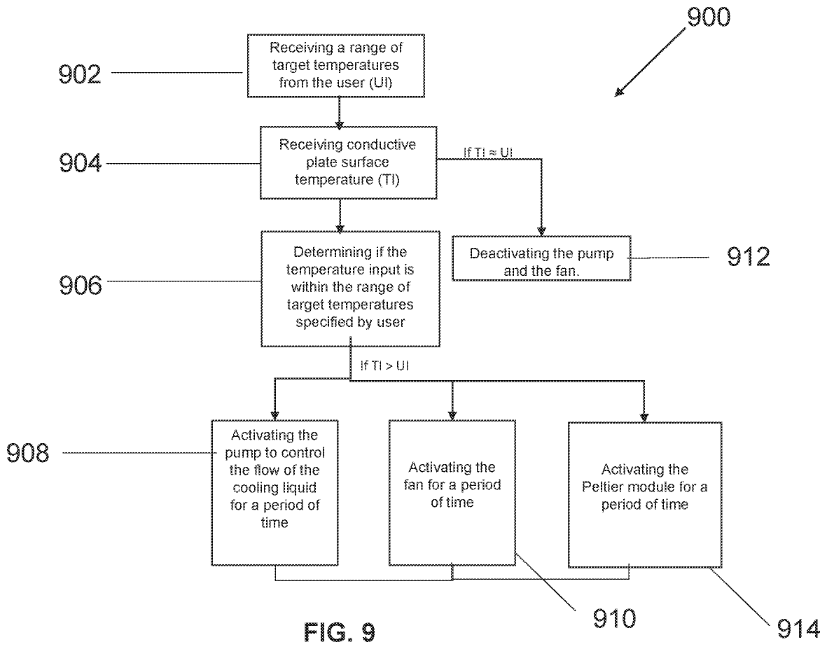

FIG. 9 is a flow diagram illustrating an exemplary method of operation 900 of the temperature control system 800. This implementation includes receiving a range of target temperatures (UI) of the surface of the conductive platform via the user interface (902). In some implementations of the temperature control system 800, the range of target temperatures from the user can be, e.g., 1-30° C., 5-20° C., or 13-17° C. (vasoconstriction in the AVAs occurs at about 12° C.). In the implementation presented in FIG. 9, the method includes receiving a temperature of the surface of the conductive plate 200 from the temperature probe 204 to determine the actual surface temperature (TI) (904). In the implementation, the method of operation 900 includes determining, via the processor 208b, if the TI is within UI (906). In the implementation presented in FIG. 9, if TI is greater than UI, the next step of the method of operation 900 includes activating the pump 708 of the liquid cooling system 700 for a time period (908) and/or activating the fan 600 (910) to increase heat dissipation from the portable cooling unit 100 and cool the surface of the conductive plate 200. In an implementation, liquid cooling system 700 and fan 600 are activated for the same time period. In some implementations of the method of operation 900, the processor 208b can be configured to only activate the fan 600 or the pump 708 at one time. In some implementations of the method of operation 900, the pump 708 can be actuated by the processor 208b to operate at a maximum speed when activated. In some implementations of the method of operation 900, the pump 708 can be actuated by the processor 208b to operate at a maximum speed for a fixed period of time when activated and can be configured to operate at different reduced speeds after the fixed period of time, depending on the temperature of the surface of the first side 202 of the conductive plate 200, e.g., to conserve power consumption from the power source 200. In an implementation of operation 900, if TI is greater than UI, the next step of the method of operation 900 includes activating Peltier module 500 alone or in combination with fan 600 and/or liquid cooling system 700 (914).

In the implementation presented in FIG. 9, if TI is within UI, the next step of the method of operation 900 includes deactivating the pump 708 of the liquid cooling system 700 and deactivating the fan 600 to maintain the TI (912). In some implementations of the method of operation 900, the processor 208b can further be configured to receive a second range of temperatures from the user representing actual core body temperature measured by an external probe (e.g., thermometer). In some implementations of the method of operation 900, the pump 708, the fan 600, and/or Peltier module 500 can be activated for a time period reduce the core body temperature by 2° C. within a time period of 5 minutes.

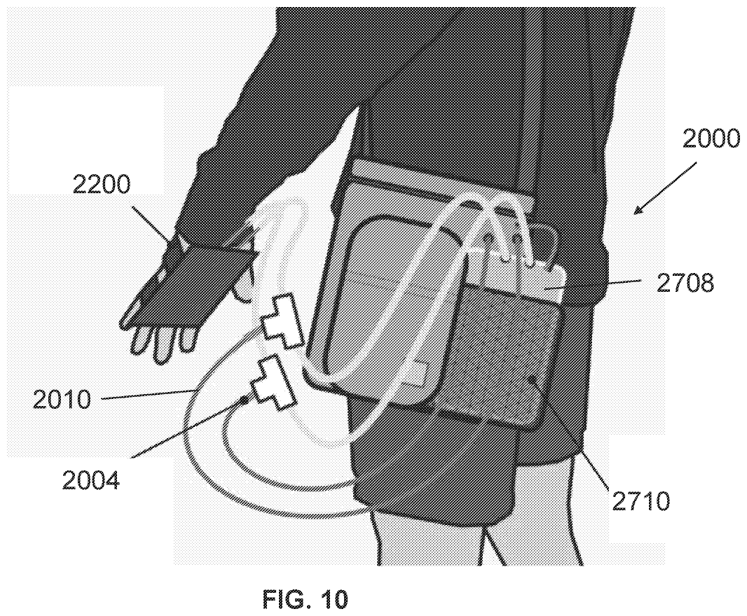

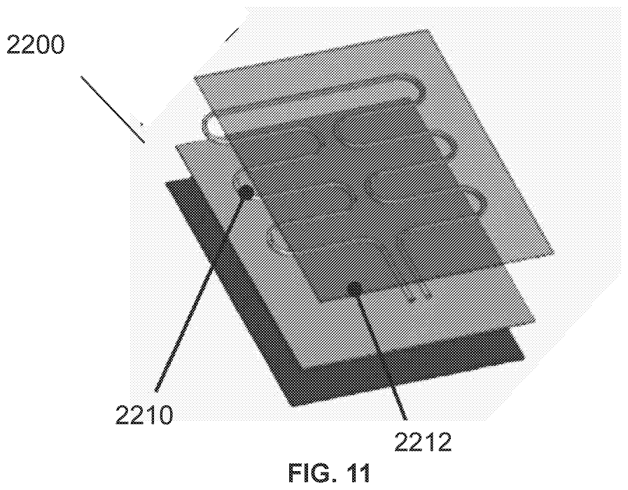

Described in this specification is a palmar cooling device 2000 as shown in FIG. 10. In an aspect, the palmar cooling device 2000 includes a handpiece 2200, a pump 2708, and a fluid reservoir 2710. In an implementation, pump 2708 and a fluid reservoir 2710 are worn in a bag or pouch on the body (e.g., abdomen, back, or side) of a used. Handpiece 2200 includes tubing 2210 formed from a conductive material (e.g., copper, aluminum, or an alloy thereof) configured to allow fluid (e.g., a liquid coolant, e.g., a refrigerant or water) to flow in a path through the handpiece in a set direction (FIG. 11). Tubing 2210 is housed in a housing 2212. In an implementation, tubing 2210 is housed in a bag or pouch, e.g., a thermoplastic polyurethane (TPU) coated nylon taffeta pouch. In an example implementation, the tubing is configured to allow a directed flow in a set path, such as a serpentine path, to allow efficient heat transfer through convection.

In some implementations, the palmar cooling device 2000 optionally include a variable-speed heat pump that regulates the flow of fluid (e.g., a liquid coolant, e.g., a refrigerant or water) through the hand piece 2200 based on temperature values obtained by one or more temperature probes 2004 disposed in or on connection tubing 2010 between pump 2708 and hand piece 2200. In an example implementation, the heat pump begins operation at full speed and, after an initial period (e.g., 1 minute), adjusts the speed of the pump based on the measured temperature differential and current flow rate. The palmar cooling device 2000 can be configured to increase the rate of cooling while minimizing power consumption.

### EXAMPLES

Described below in this specification are example aspects/implementations of the technologies for a portable cooling unit including a conducting plate, a heat sink in combination with heat pipes and/or a Peltier module as described above.

FIG. 12 shows a perspective schematic view of an exemplary portable cooling unit 1000. The portable cooling unit 1000 presented in FIG. 12 is designed to interact with a palm of a user and reduce core temperature via the concept of palmar cooling. FIG. 12 presents structural components of the portable cooling unit 1000 including the technologies described above in the specification for portable cooling unit 100 and illustrated in FIGS. 1-7. These include a conductive plate 200, a heat sink 400, one or more heat pipes 300 thermally connecting the conductive plate 200 and the heat sink 400, a power source 120, and a fan 600. As shown in FIG. 12, the fan 600 is coupled to the heat sink 400 and electrically connected to the power source 120 via a connecting cable 110.

In an implementation, the portable cooling unit 1000 includes six heat pipes 300, each thermally connecting the conductive plate 200 and the heat sink 400. In some implementations of the portable cooling unit 1000, the portable cooling unit 1000 can include between two and thirty, or more, heat pipes 300, each thermally connecting the conductive plate 200 and the heat sink 400.

In the portable cooling unit 1000, the heat sink 400 of the portable cooling unit 1000 includes a plurality of fins 408 to maximize the surface area for heat dissipation from the heat sink 400 into the ambient air. In the portable cooling unit 1000, the heat sink 400 dissipates the heat via the plurality of fins 408 and the lower side 404 of the heat sink 400 via radiation, conduction, convection, or a combination of at least two of the processes of heat dissipation. In an implementation of portable cooling unit 1000, the six heat pipes 300 and the heat sink 400 are configured to dissipate heat away from the user at a rate at which the user can restore normal core body temperature within 10 minutes of usage of the portable cooling unit 1000.

The portable cooling unit 1000 includes an implementation of the fan 600 shown in FIG. 6 comprising a motor 604, fan blades 602 actuated by the motor 604, a housing 606, an air inlet 608 and an air outlet 610. In portable cooling unit 1000, the fan 600 is positioned in a way that the air outlet 610 of the fan 600 is facing the lower side 404 of the heat sink 400 to cool down the heat sink 400 when the fan blades 602 are actuated. In portable cooling unit 1000, the fan 600 is powered by the power source 120 electrically connected to the power source 120 via a connecting cable 110.

In the portable cooling unit 1000, the first side 202 of the conductive plate 200 is attached to connecting straps 1102 to secure the palm of the user to the first side 202 of the conductive plate 200. In the portable cooling unit 1000, the portable cooling unit 1000 includes at least one wrist strap 1104 for securing the power source 120 to the wrist of the user to power the portable cooling unit 1000. In some implementations of the portable cooling unit 1000, the portable cooling unit can include a body connecting strap (not shown in FIG. 12) for connecting the power source 120 to a part of the body of the user other than the wrist. In some implementations of the portable cooling unit 1000, the connecting straps 1102 and wrist straps 1104 can be made from nylon taffeta or velcro.

In the portable cooling unit 1000, the power source 120 disposed on the wrist of the user is a battery unit capable of powering the different components of the portable cooling unit 1000. In some implementations of the portable cooling unit 1000, the power source 120 disposed on the wrist of the user is a battery unit capable of powering the different components of the portable cooling unit 1000, e.g., for over an hour or more of continuous usage of the portable cooling unit 1000. In some implementations, power source 120 is disposed elsewhere on the body (e.g., in a pack disposed on the user's back, side, or abdomen).

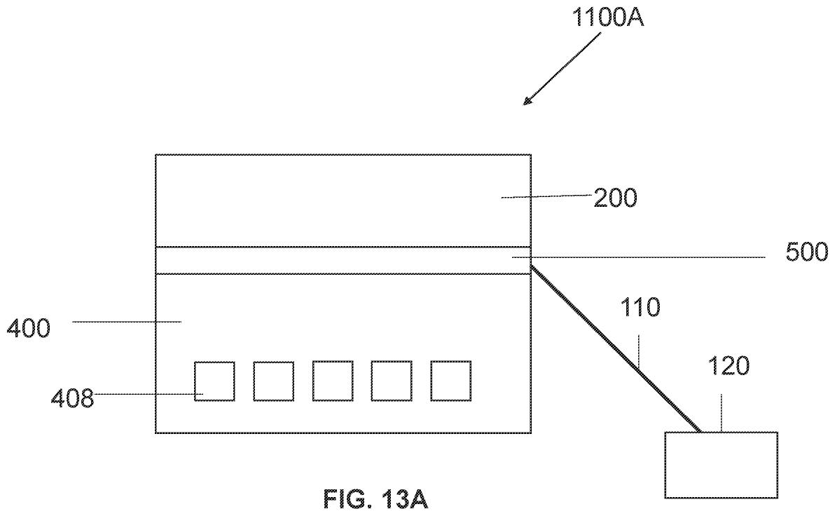

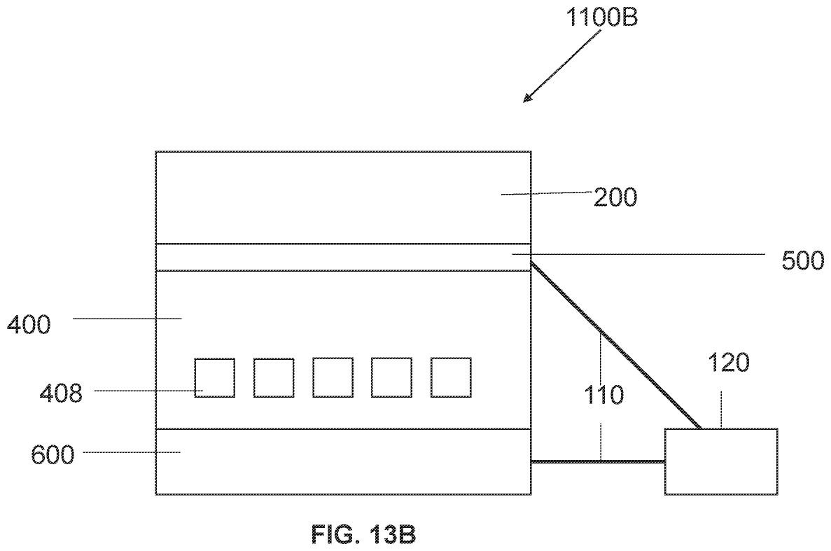

FIGS. 13A-13B show schematic diagrams of exemplary implementations of a portable cooling unit as described in this specification (portable cooling units 1100A and 1100B). In the portable cooling unit 1100A presented in FIG. 13A, the portable cooling unit 1100A does not include heat pipes 300. In the portable cooling unit 1100A presented in FIG. 13A, the portable cooling unit 1100A is designed to interact with a palm of a user and reduce core temperature via the concept of palmar cooling. FIG. 13A also presents structural components of the portable cooling unit 1100A including the technologies described above in the specification and illustrated in FIGS. 1-7. These include a conductive plate 200, a heat sink 400, a Peltier module 500 thermally connecting the conductive plate 200 and the heat sink 400, and a power source 120. In the implementation presented in FIG. 13B, the portable cooling unit 1100A presented in FIG. 13A further includes the fan 600 coupled to the heat sink 400 and electrically connected to the power source 120 via a connecting cable 110.

In the portable cooling unit 1100A presented in FIG. 13A, the Peltier module 500 includes a heat absorbing end 502, a heat releasing end 504, and a plurality of semiconductor elements 506 connected to the power source 120 and thermally connecting the heat absorbing end 502 and the heat releasing end 504. In the portable cooling unit 1100A, the heat absorbing end 502 of the Peltier module 500 is thermally connected to the second side 206 of the conductive plate 200 to receive the heat from the conductive plate 200. In the portable cooling unit 1100A, the heat releasing end 504 of the Peltier module 500 is disposed on the upper side 402 of the heat sink 400 to release heat via the heat sink 400 for dissipation into the ambient air.

In the portable cooling unit 1100A, the heat sink 400 of the portable cooling unit 1100A includes a plurality of fins, e.g., fins 408, to maximize the surface area for heat dissipation from the heat sink 400. In this implementation, the heat sink 400 can dissipate the heat via the plurality of fins 408 and the lower side 404 of the heat sink 400 via radiation, conduction, convection, or a combination of at least two of the processes of heat dissipation. In an example implementation, the heat sink 400 can be cooled by the ambient air.

In the implementation presented in FIG. 13B (portable cooling unit 1100B), the portable cooling unit 1100A of FIG. 13A further includes an implementation of the fan 600 shown in FIG. 6 comprising a motor 604, fan blades 602 actuated by the motor 604, a housing 606, an air inlet 608 and an air outlet 610. In this portable cooling unit 1100B, the fan 600 is positioned in a way that the air outlet of the fan 600 is facing the lower side 404 of the heat sink 400 to cool down the heat sink 400 when the fan blades 602 are actuated. In this portable cooling unit 1100B, the fan 600 is powered by the power source 120 electrically connected to the power source 120 via a connecting cable 110. In some implementations, fan 600 is mounted on a side other than lower side 404.

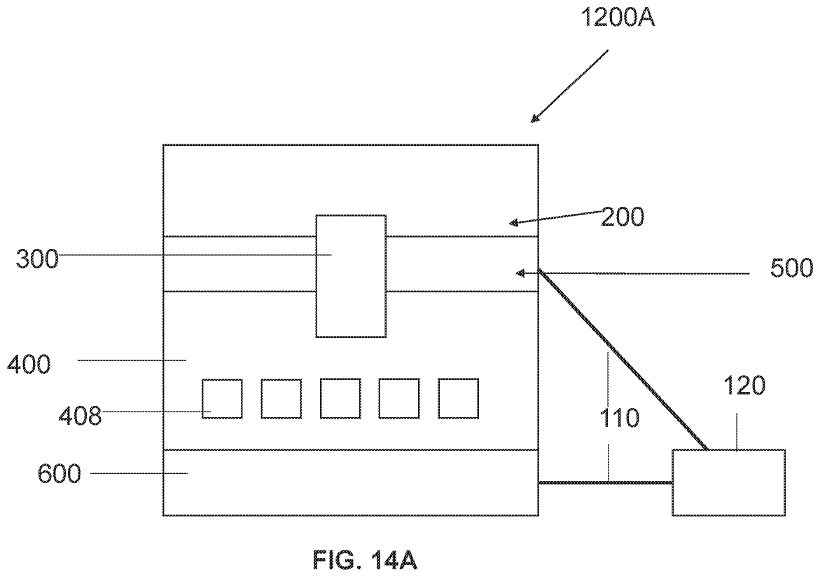

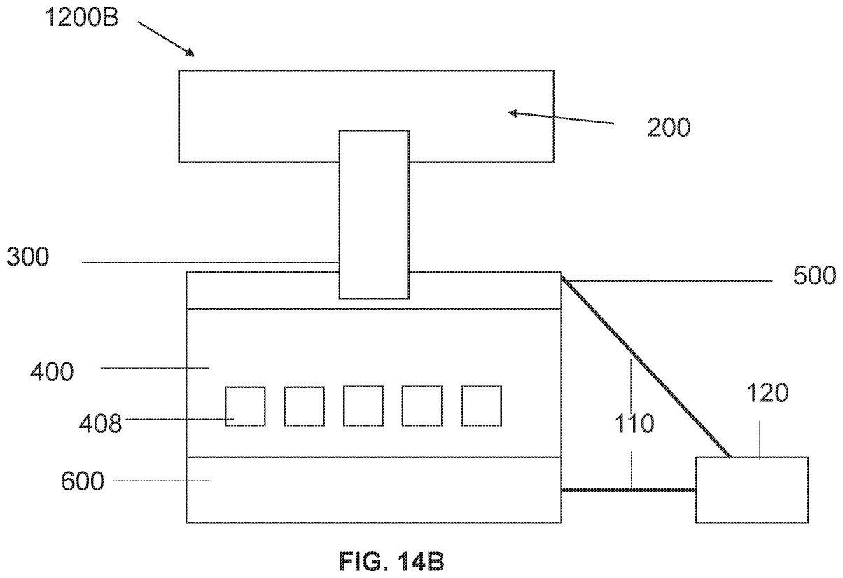

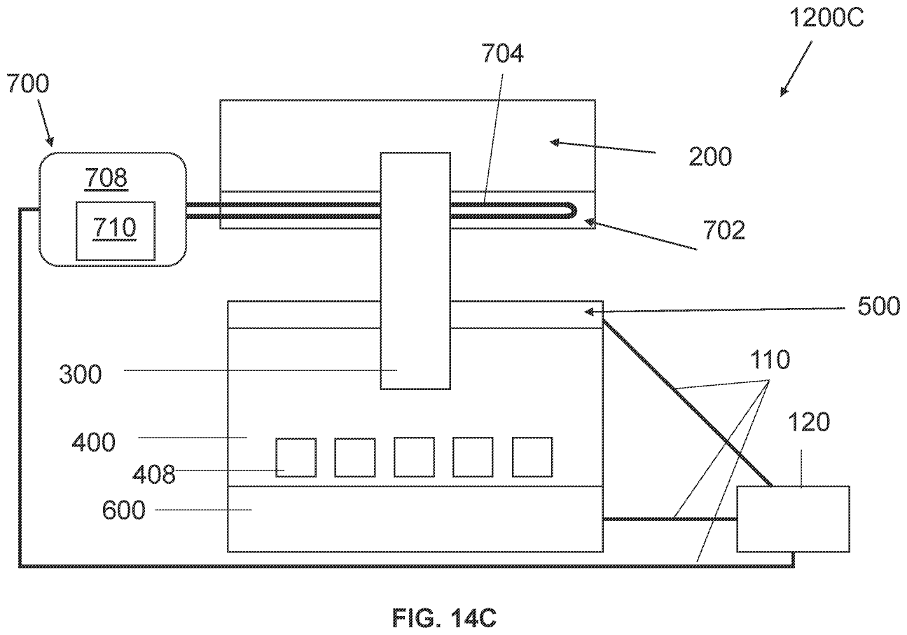

FIGS. 14A-14C shows schematic diagrams of exemplary implementations of a portable cooling unit as described in this specification (portable cooling units 1200A, 1200B, 1200C). In the portable cooling units 1200A, 1200B, 1200C presented in FIG. 14A-14C, the portable cooling unit 1200A, 1200B, 1200C is designed to interact with a palm of a user and reduce core temperature via the concept of palmar cooling. FIG. 14A illustrates structural components of the portable cooling units 1200A, 1200B, 1200C including the technologies described above in the specification and in FIGS. 1-7. These include a conductive plate 200, a heat sink 400, one or more heat pipes 300, a Peltier module 500, a power source 120, and a fan 600.

In the portable cooling unit 1200A, 1200B, 1200C presented in FIGS. 14A-14C, the portable cooling unit 1200A, 1200B, 1200C includes at least one heat pipe 300 thermally connecting the conductive plate 200 and the heat sink 400 and/or Peltier module 500. In some implementations of the portable cooling unit 1200A, 1200B, 1200C, the portable cooling unit can include six heat pipes 300, each thermally connecting the conductive plate 200 and the heat sink 400 and/or Peltier module 500. In other implementations of the portable cooling unit 1200A, 1200B, 1200C, the portable cooling unit 1200A, 1200B, 1200C can include between two and thirty, or more, heat pipes 300, each thermally connecting the conductive plate 200 and the heat sink 400 and/or Peltier module 500.

In the portable cooling unit 1200A, 1200B, 1200C presented in FIGS. 14A-14C, the heat sink 400 of the portable cooling unit 1200A, 1200B, 1200C includes a plurality of fins 408 to maximize the surface area for heat dissipation from the heat sink 400. In the portable cooling unit 1200A, 1200B, 1200C, the heat sink 400 can dissipate the heat via the plurality of fins 408 and the lower side 404 of the heat sink 400 via radiation, conduction, convection, or a combination of at least two of the processes of heat dissipation. In the portable cooling unit 1200A, 1200B, 1200C presented in FIGS. 14A-14C, the heat pipes 300, the heat sink 400, and/or Peltier module 500 are configured to dissipate heat away from the user at a rate at which the user can restore normal core body temperature within 10 minutes of usage of the portable cooling unit 1200A, 1200B, 1200C.

In the portable cooling unit 1200A, 1200B, 1200C presented in FIGS. 14A-14C, the portable cooling unit 1200A, 1200B, 1200C includes an implementation of the fan 600 shown in FIG. 6 comprising a motor 604, fan blades 602 actuated by the motor 604, a housing 606, an air inlet 608 and an air outlet 610. In the portable cooling unit 1200A, 1200B, 1200C, the fan 600 is positioned in a way that the air outlet of the fan 600 is facing the lower side 404 of the heat sink 400 to cool down the heat sink 400 when the fan blades 602 are actuated. In the portable cooling unit 1200A, 1200B, 1200C, the fan 600 is powered by the power source 120 and can be electrically connected to the power source 120 via a connecting cable 110.

In the portable cooling unit 1200A presented in FIG. 14A, the Peltier module 500 includes a heat absorbing end 502, a heat releasing end 504, and a plurality of semiconductor elements 506 connected to the power source 120 and thermally connecting the heat absorbing end 502 and the heat releasing end 504. In the portable cooling unit 1200A presented in FIG. 14A, the heat absorbing end 502 of the Peltier module 500 is thermally connected to the second side 206 of the conductive plate 200 and the heat pipes 300 to receive the heat from the conductive plate 200 and/or the heat pipes 300. In the implementations presented in FIGS. 14B and 14C, the heat absorbing end 502 of the Peltier module 500 is not thermally connected to the second side 206 of the conductive plate 200. In portable cooling unit 1200B (FIG. 14B), the Peltier module 500 receives heat at heat absorbing end 502 from the heat pipes 300 only (at cold interface 304). In the 1200A, 1200B, 1200C presented in FIG. 14A-14C, the heat releasing end 504 of the Peltier module is disposed on the upper side of the heat sink 400 to release the received heat via the heat sink 400 for dissipation into the ambient air.

In portable cooling unit 1200C presented in FIG. 14C, the portable cooling unit 1200C includes a liquid cooling system 700. The liquid cooling system 700 can be used to increase the efficiency of the portable cooling unit 1200C by increasing a rate of heat transfer through the conductive plate 200. In the implementation of the liquid cooling system 700 shown in FIG. 14C, the liquid cooling system 700 includes an internal fluid chamber 702 containing conductive tubing 704, a pump 708 connected to the conductive tubing via fluid ports 706, where the pump 708 is electrically connected to the power source 120 via a connecting cable 110. In the same implementation of the liquid cooling system 700, the pump includes a reservoir 710 containing a liquid coolant (e.g., a refrigerant) for flowing through the conductive tubing 704.

In the implementation of the liquid cooling system 700 shown in FIG. 14C, the internal fluid chamber 702 is disposed on the second side 206 of the conductive plate 200. The liquid coolant flowing through the conductive tubing 704 i configured to receive the heat from the conductive plate 200 and actively cool the conductive plate 200. In some implementations of the liquid cooling system 700, the internal fluid chamber 702 could be made from a thermally conductive material such as copper. In some implementations of the liquid cooling system 700, the conductive tubing 704 is serpentine-shaped to increase surface area for absorbing the heat from the conductive plate 200. In some implementations of the liquid cooling system 700, the liquid coolant can be a heat sensitive phase-change liquid, including, e.g., water, a hydrocarbon, a chlorofluorocarbon, a fluorocarbon (e.g., perfluoropentane), a salt hydrate, or a combination thereof. In some implementations of the liquid cooling system 700, the liquid coolant can include water.

While the advantages and preferred implementations of the present technologies have been described in this specification, it should be understood by those skilled in the art that the above are merely several illustrative implementations of the present technologies without limiting the scope thereof, wherein various modifications, alterations or substitutions may be made to the specific components of the implementations without departing from the spirit and scope of the technologies and the claims.

Item 1. A portable cooling unit, comprising: a conductive plate having a first side and a second side; a heat sink having an upper side, a lower side, and a body thermally connecting the upper side and the lower side, the upper side of the heat sink positioned facing the second side of the conductive plate; a fan having an air outlet side and an air inlet side, the fan connected to the heat sink such that the air outlet side faces the lower side of the heat sink; a power source connected to the fan via at least one connecting cable; and one or more heat pipes thermally connecting the conductive plate to the heat sink.

Item 2. The portable cooling unit according to item 1, wherein the first side of the conductive plate is configured to interface with a palm of a user.

Item 3. The portable cooling unit according to item 2, wherein the first side of the conductive plate is configured to be secured with the palm of the user via at least one connecting strap.

Item 4. The portable cooling unit according to any one of the preceding items, wherein the power source is a battery configured to be secured to a wrist of the user via at least one wrist strap.

Item 5. The portable cooling unit according to any one of items 1-3, wherein the power source is a battery positioned on a part of the user's body other than the wrist of the user.

Item 6. The portable cooling unit according to any one of the preceding items, further comprising at least one temperature probe disposed on the first side of the conductive plate, the at least one temperature probe configured to measure a surface temperature of the first side of the conductive plate.

Item 7. The portable cooling unit according to any one of the preceding items, further comprising a liquid cooling system comprising: an internal fluid chamber thermally connected to the second side of the conductive plate, the internal fluid chamber comprising conductive tubing; and a pump fluidically connected to the internal fluid chamber via at least one fluid port, the pump configured to circulate a coolant through the conductive tubing of the internal fluid chamber, wherein the pump is electrically connected to the power source via at least one connecting cable, and wherein the coolant is configured to remove heat from the conductive plate.

Item 8. The portable cooling unit according to item 7, wherein the conductive tubing of the internal fluid chamber is serpentine shaped.

Item 9. The portable cooling unit according to item 7 or item 8, wherein the conductive tubing is made from copper.

Item 10. The portable cooling unit according to any one of items 6-9, further comprising a user interface configured for interaction with the user, the user interface comprising: a graphic user interface (GUI) disposed on the conductive plate; a processor coupled with the GUI; a memory coupled with and readable by the processor and storing therein a set of instructions which, when executed by the processor, causes the processor to perform operations comprising: (a) receiving a user input via the GUI, the user input comprising a range of target surface temperatures of the first side of the conductive plate for maintaining a target core body temperature of the user; (b) receiving the surface temperature of the first side of the conductive plate from the at least one temperature probe; (c) determining if the surface temperature of the first side of the conductive plate is within the range of target surface temperature; and if the surface temperature of the first side of the conductive plate is greater than the range of target surface temperature, (d)activating the pump to control a flow rate of the coolant flowing through the internal fluid chamber for a first period of time; (e) activating the fan for a second period of time; (f) repeating steps (b)-(e) until the surface temperature of the first side of the conductive plate is within the range of target surface temperature; and (g) deactivating the pump and the fan when the surface temperature of the first side of the conductive plate is within the range of target surface temperature.

Item 11. The portable cooling unit according to any one of the preceding items, further comprising a cooling element thermally connected to the heat sink, the cooling element configured to remove heat from the heat sink.

Item 12. The portable cooling unit according to item 11, wherein the cooling element comprises a phase-change material configured to cool the heat sink.

Item 13. The portable cooling unit according to item 11, wherein the cooling element is a Peltier module electrically connected to the power source via at least one connecting cable, the Peltier module comprising: a heat absorbing end configured for absorbing heat; a heat releasing end configured to release the absorbed heat; and a plurality of semiconductor elements electrically connected to the power source, the plurality of semiconductor elements thermally connecting the heat absorbing end and the heat releasing end.

Item 14. The portable cooling unit according to item 13, wherein the heat absorbing end and the heat releasing end each comprise: a conductive layer thermally connected with the plurality of semiconductor elements; and an insulative layer disposed on each of the conductive layers.

Item 15. The portable cooling unit according to any one of the preceding items, wherein the heat sink comprises a plurality of fins configured to increase a surface area of the heat sink for heat dissipation.

Item 16. The portable cooling unit according to any one of the preceding items, wherein heat dissipation from the heat sink occurs via at least one of radiation, conduction, or convection.

Item 17. The portable cooling unit according to any one of the preceding items, wherein the conductive plate further comprises an insulation layer disposed on the first side of the conductive plate, the insulative layer configured to prevent loss of heat from the first side of the conductive plate.

Item 18. The portable cooling unit according to any one of the preceding items, wherein the one or more heat pipes are made from a thermally conductive material.

Item 19. The portable cooling unit according to any one of the preceding items, wherein the one or more heat pipes comprise between two and thirty heat pipes.

Item 20. The portable cooling unit according to any one of the preceding items, wherein the one or more heat pipes comprise six heat pipes.

Item 21. The portable cooling unit according to any one of the preceding items, wherein each of the one or more heat pipes comprises: a hot interface thermally connected to the conductive plate; a cold interface thermally connected to the heat sink; a conductive tunnel connecting the hot interface and the cold interface; and a volatile liquid disposed at the hot interface, the volatile liquid configured to remove the heat from the conductive plate and phase-change to a vapor.

Item 22. The portable cooling unit of item 21, wherein: the cold interface is configured to receive and condense the vapor to the volatile liquid; and the conductive tunnel is configured to transport the condensed volatile liquid from the cold interface to the hot interface.

Item 23. The portable cooling unit of item 21, wherein the conductive tunnel comprises an interior surface configured to transport of the condensed volatile liquid via capillary action.

Item 24. A portable cooling unit, comprising: a conductive plate having a first side and a second side, the first side of the conductive plate configured to interface with a palm of a user and configured to be secured with the palm of the user via at least one connecting strap; a heat sink having an upper side, a lower side, and a body thermally connecting the upper side and the lower side, the upper side of the heat sink positioned facing the second side of the conductive plate, the heat sink comprising a plurality of fins disposed on the body, the plurality of fins being configured to increase a surface area of the heat sink for heat dissipation; a fan having an air outlet side and an air inlet side, the fan connected to the heat sink such that the air outlet side faces the lower side of the heat sink; a power source connected to the fan via at least one connecting cable and configured to be secured to a wrist of the user via at least one wrist strap; and one or more heat pipes thermally connecting the conductive plate to the heat sink.

Item 25. A portable cooling unit, comprising: a conductive plate having a first side and a second side; a heat sink having an upper side, a lower side, and a body thermally connecting the upper side and the lower side, the upper side of the heat sink positioned facing the second side of the conductive plate, the heat sink comprising a plurality of fins disposed on the body, the plurality of fins being configured to increase a surface area of the heat sink for heat dissipation; a power source; and a Peltier module electrically connected to the power source via at least one connecting cable, the Peltier Module comprising: a heat absorbing end thermally connected to the second side of the conductive plate and configured to absorb heat from the conductive plate; a heat releasing end thermally connected to the upper side of the heat sink and configured release the absorbed heat from the conductive plate into the heat sink; and a plurality of semiconductor elements electrically connected to the power source, the plurality of semiconductor elements thermally connecting the heat absorbing end and the heat releasing end.

Item 26. The portable cooling unit according to item 25, further comprising a fan having an air outlet side and an air inlet side, the fan connected to the heat sink such that the air outlet side is positioned facing the lower side of the heat sink, and the power source connected to the fan via at least one connecting cable.

Item 27. The portable cooling unit according item 25 or item 26, wherein the first side of the conductive plate is configured to interface with a palm of a user.

Item 28. The portable cooling unit according to item 27, wherein the first side of the conductive plate is configured to be secured with the palm of the user via at least one connecting strap.

Item 29. The portable cooling unit according to any one of items 25-28, wherein the power source is a battery configured to be secured to a wrist of the user via at least one wrist strap.

Item 30. The portable cooling unit according to any one of items 25-28, wherein the power source is positioned on a part of the user's body other than the wrist of the user.

Item 31. The portable cooling unit according to any one of items 25-30, further comprising at least one temperature probe disposed on the first side of the conductive plate, the at least one temperature probe configured to measure a surface temperature of the first side of the conductive plate.

Item 32. The portable cooling unit according to item 31, further comprising a user interface configured for interaction with the user, the user interface comprising: a graphic user interface (GUI) disposed on the conductive plate; a processor coupled with the GUI; a memory coupled with and readable by the processor and storing therein a set of instructions which, when executed by the processor, causes the processor to perform operations comprising: (a) receiving a user input via the GUI, the user input comprising a range of target surface temperatures of the first side of the conductive plate for maintaining a target core body temperature of the user; (b) receiving the surface temperature of the first side of the conductive plate from the at least one temperature probe; (c) determining if the surface temperature of the first side of the conductive plate is within the range of target surface temperature; and if the surface temperature of the first side of the conductive plate is greater than the range of target surface temperature, (d) activating the Peltier module or the fan, or both, for a period of time; (e) repeating steps (b)-(d) until the surface temperature of the first side of the conductive plate is within the range of target surface temperature; and (f) deactivating the fan when the surface temperature of the first side of the conductive plate is within the range of target surface temperature.

Item 33. The portable cooling unit according to any one of items 25-32, wherein heat dissipation from the heat sink occurs via at least one of radiation, conduction, or convection.

Item 34. The portable cooling unit according to any one of items 25-33, wherein the conductive plate further comprises an insulation layer disposed on the first side of the conductive plate, the insulative layer configured to prevent loss of heat from the first side of the conductive plate.

Item 35. A portable cooling unit, comprising: a conductive plate having a first side and a second side; a heat sink having an upper side, a lower side, and a body thermally connecting the upper side and the lower side, the upper side of the heat sink positioned to facing the second side of the conductive plate, the heat sink comprising a plurality of fins disposed on the body, the plurality of fins being configured to increase a surface area of the heat sink for heat dissipation; a fan having an air outlet side and an air inlet side, the fan connected to the heat sink such that the air outlet side is positioned faces the lower side of the heat sink, a power source connected to the fan via at least one connecting cable; one or more heat pipes thermally connecting the conductive plate to the heat sink; and a Peltier module electrically connected to the power source via at least one connecting cable, the Peltier Module comprising: a heat absorbing end thermally connected to the second side of the conductive plate and configured to absorb heat from the conductive plate; a heat releasing end thermally connected to the upper side of the heat sink and configured release the absorbed heat from the conductive plate into the heat sink; and a plurality of semiconductor elements electrically connected to the power source, the plurality of semiconductor elements thermally connecting the heat absorbing end and the heat releasing end.

Item 36. The portable cooling unit of item 35, wherein: the heat absorbing end of the Peltier module is thermally connected to the one or more heat pipes and configured to absorb heat from the one or more heat pipes; and the heat releasing end of the Peltier module is configured to release the absorbed hear from the one or more heat pipes into the heat sink.

Item 37. A portable cooling unit, comprising: a conductive plate having a first side and a second side; a heat sink having an upper side, a lower side, and a body thermally connecting the upper side and the lower side, the upper side of the heat sink positioned facing the second side of the conductive plate, the heat sink comprising a plurality of fins disposed on the body, the plurality of fins being configured to increase a surface area of the heat sink for heat dissipation; a fan having an air outlet side and an air inlet side, wherein the fan is connected to the heat sink such that the air outlet side is positioned to face the lower side of the heat sink, a power source connected to the fan via at least one connecting cable; one or more heat pipes; and a Peltier module electrically connected to the power source via at least one connecting cable, the Peltier Module comprising: a heat absorbing end thermally connected to the one or more heat pipes and configured to absorb heat from the one or more heat pipes; a heat releasing end thermally connected to the upper side of the heat sink and configured release the absorbed heat from the one or more heat pipes into the heat sink; and a plurality of semiconductor elements electrically connected to the power source, the plurality of semiconductor elements thermally connecting the heat absorbing end and the heat releasing end.

Item 38. The portable cooling unit according to item 37, wherein the first side of the conductive plate is configured to interface with a palm of a user.

Item 39. The portable cooling unit according to item 38, wherein the first side of the conductive plate is configured to be secured with the palm of the user via at least one connecting strap.

Item 40. The portable cooling unit according to any one of items 37-39, wherein the power source is a battery configured to be secured to a wrist of the user via at least one wrist strap.

Item 41. The portable cooling unit according to any one of items 37-39, wherein the power source is a battery positioned on a part of the user's body other than the wrist of the user.

Item 42. The portable cooling unit according to any one of items 37-41, further comprising at least one temperature probe disposed on the first side of the conductive plate, the at least one temperature probe configured to measure a surface temperature of the first side of the conductive plate.

Item 43. The portable cooling unit according to item 42, further comprising a user interface configured for interaction with the user, the user interface comprising: a graphic user interface (GUI) disposed on the conductive plate; a processor coupled with the GUI; a memory coupled with and readable by the processor and storing therein a set of instructions which, when executed by the processor, causes the processor to perform operations comprising: (a) receiving a user input via the GUI, the user input comprising a range of target surface temperatures of the first side of the conductive plate for maintaining a target core body temperature of the user; (b) receiving the surface temperature of the first side of the conductive plate from the at least one temperature probe; (c) determining if the surface temperature of the first side of the conductive plate is within the range of target surface temperature; and if the surface temperature of the first side of the conductive plate is greater than the range of target surface temperature, (d) activating the Peltier module or the fan, or both, for a period of time; (e) repeating steps (b)-(d) until the surface temperature of the first side of the conductive plate is within the range of target surface temperature; and (f) deactivating the pump and the fan when the surface temperature of the first side of the conductive plate is within the range of target surface temperature.

Item 44. The portable cooling unit according to any one of items 37-43, wherein heat dissipation from the heat sink occurs via at least one of radiation, conduction, or convection.

Item 45. The portable cooling unit according to any one of items 37-44, wherein the conductive plate further comprises an insulation layer disposed on the first side of the conductive plate, the insulative layer configured to prevent loss of heat from the first side of the conductive plate.

Item 46. The portable cooling unit according to any one of items 37-45, wherein the one or more heat pipes are made from a thermally conductive material.

Item 47. The portable cooling unit according to any one of items 37-46, wherein the one or more heat pipes comprise between two and thirty heat pipes.

Item 48. The portable cooling unit according to any one of items 37-47, wherein the one or more heat pipes comprise six heat pipes.

Item 49. The portable cooling unit according to any one of items 37-48, wherein each of the one or more heat pipes comprises: a hot interface thermally connected to the conductive plate; a cold interface thermally connected to the Peltier module; a conductive tunnel connecting the hot interface and the cold interface; and a volatile liquid disposed at the hot interface, the volatile liquid configured to remove the heat from the conductive plate and phase-change to a vapor.

Item 50. The portable cooling unit of item 49, wherein: the cold interface is configured to receive and condense the vapor to the volatile liquid; and the conductive tunnel is configured to transport the condensed volatile liquid from the cold interface to the hot interface.

Item 51. The portable cooling unit of item 50, wherein the conductive tunnel comprises an interior surface configured to transport of the condensed volatile liquid via capillary action.

Item 52. A portable cooling unit, comprising: a conductive plate having a first side and a second side; a heat sink having an upper side, a lower side, and a body thermally connecting the upper side and the lower side, the upper side of the heat sink positioned facing the second side of the conductive plate, the heat sink comprising a plurality of fins disposed on the body, the plurality of fins being configured to increase a surface area of the heat sink for heat dissipation; a fan connected to the power source via at least one connecting cable, the fan having an air outlet side and an air inlet side, the fan connected to the heat sink such that the air outlet side is positioned facing the lower side of the heat sink; a power source connected to the fan via at least one connecting; one or more heat pipes thermally connecting the conductive plate to the heat sink; a liquid cooling system comprising: an internal fluid chamber thermally connected to the second side of the conductive plate, the internal fluid chamber comprising conductive tubing; and a pump electrically connected to the power source via at least one connecting cable and fluidically connected to the internal fluid chamber via at least one fluid port, the pump configured to circulate a coolant through the conductive tubing of the internal fluid chamber, the coolant configured to remove heat from the conductive plate via convection; and a Peltier module electrically connected to the power source via at least one connecting cable, the Peltier Module comprising: a heat absorbing end thermally connected to the one or more heat pipes and configured to absorb heat from the one or more heat pipes; a heat releasing end thermally connected to the upper side of the heat sink and configured release the absorbed heat from the one or more heat pipes into the heat sink; and a plurality of semiconductor elements electrically connected to the power source, the plurality of semiconductor elements thermally connecting the heat absorbing end and the heat releasing end.

Item 53. The portable cooling unit according to item 52, wherein the first side of the conductive plate is configured to interface with a palm of a user.

Item 54. The portable cooling unit according to item 53, wherein the first side of the conductive plate is configured to be secured with the palm of the user via at least one connecting strap.

Item 55. The portable cooling unit according to item 53, wherein the power source is configured to be secured to a wrist of the user via at least one wrist strap.

What is claimed is:

1. A portable cooling unit, comprising:
a conductive plate having a first side and a second side;
a heat sink having an upper side, a lower side, and a body thermally connecting the upper side and the lower side, the upper side of the heat sink positioned facing the second side of the conductive plate;
a fan having an air outlet side and an air inlet side, the fan connected to the heat sink such that the air outlet side faces the lower side of the heat sink;
a power source connected to the fan via at least one connecting cable; and
one or more heat pipes thermally connecting the conductive plate to the heat sink.

2. The portable cooling unit according to claim 1, wherein the first side of the conductive plate is configured to interface with a palm of a user.

3. The portable cooling unit according to claim 2, wherein the first side of the conductive plate is configured to be secured with the palm of the user via at least one connecting strap.

4. The portable cooling unit according to any one of the preceding claims, wherein the power source is a battery configured to be secured to a wrist of the user via at least one wrist strap.

5. The portable cooling unit according to any one of claims 1-3, wherein the power source is a battery positioned on a part of the user's body other than the wrist of the user.

6. The portable cooling unit according to any one of the preceding claims, further comprising at least one temperature probe disposed on the first side of the conductive plate, the at least one temperature probe configured to measure a surface temperature of the first side of the conductive plate.

7. The portable cooling unit according to any one of the preceding claims, further comprising a liquid cooling system comprising:
an internal fluid chamber thermally connected to the second side of the conductive plate, the internal fluid chamber comprising conductive tubing; and
a pump fluidically connected to the internal fluid chamber via at least one fluid port, the pump configured to circulate a coolant through the conductive tubing of the internal fluid chamber,
wherein the pump is electrically connected to the power source via at least one connecting cable, and
wherein the coolant is configured to remove heat from the conductive plate.

8. The portable cooling unit according to claim 7, wherein the conductive tubing of the internal fluid chamber is serpentine shaped.

9. The portable cooling unit according to claim 7 or claim 8, wherein the conductive tubing is made from copper.

10. The portable cooling unit according to any one of claims 6-9, further comprising a user interface configured for interaction with the user, the user interface comprising:
a graphic user interface (GUI) disposed on the conductive plate;
a processor coupled with the GUI;
a memory coupled with and readable by the processor and storing therein a set of instructions which, when executed by the processor, causes the processor to perform operations comprising:
(a) receiving a user input via the GUI, the user input comprising a range of target surface temperatures of the first side of the conductive plate for maintaining a target core body temperature of the user;
(b) receiving the surface temperature of the first side of the conductive plate from the at least one temperature probe;
(c) determining if the surface temperature of the first side of the conductive plate is within the range of target surface temperature; and
if the surface temperature of the first side of the conductive plate is greater than the range of target surface temperature,
(d) activating the pump to control a flow rate of the coolant flowing through the internal fluid chamber for a first period of time;
(e) activating the fan for a second period of time;
(f) repeating steps (b)-(e) until the surface temperature of the first side of the conductive plate is within the range of target surface temperature; and
(g) deactivating the pump and the fan when the surface temperature of the first side of the conductive plate is within the range of target surface temperature.

11. The portable cooling unit according to any one of the preceding claims, further comprising a cooling element thermally connected to the heat sink, the cooling element configured to remove heat from the heat sink.

12. The portable cooling unit according to claim 11, wherein the cooling element comprises a phase-change material configured to cool the heat sink.

13. The portable cooling unit according to claim 11, wherein the cooling element is a Peltier module electrically connected to the power source via at least one connecting cable, the Peltier module comprising:
a heat absorbing end configured for absorbing heat;
a heat releasing end configured to release the absorbed heat; and
a plurality of semiconductor elements electrically connected to the power source, the plurality of semiconductor elements thermally connecting the heat absorbing end and the heat releasing end.

14. The portable cooling unit according to claim 13, wherein the heat absorbing end and the heat releasing end each comprise:
a conductive layer thermally connected with the plurality of semiconductor elements; and
an insulative layer disposed on each of the conductive layers.

15. The portable cooling unit according to any one of the preceding claims, wherein the heat sink comprises a plurality of fins configured to increase a surface area of the heat sink for heat dissipation.

16. The portable cooling unit according to any one of the preceding claims, wherein heat dissipation from the heat sink occurs via at least one of radiation, conduction, or convection.

17. The portable cooling unit according to any one of the preceding claims, wherein the conductive plate further comprises an insulation layer disposed on the first side of the conductive plate, the insulative layer configured to prevent loss of heat from the first side of the conductive plate.

18. The portable cooling unit according to any one of the preceding claims, wherein the one or more heat pipes are made from a thermally conductive material.

19. The portable cooling unit according to any one of the preceding claims, wherein the one or more heat pipes comprise between two and thirty heat pipes.

20. The portable cooling unit according to any one of the preceding claims, wherein the one or more heat pipes comprise six heat pipes.

21. The portable cooling unit according to any one of the preceding claims, wherein each of the one or more heat pipes comprises:
a hot interface thermally connected to the conductive plate;
a cold interface thermally connected to the heat sink;
a conductive tunnel connecting the hot interface and the cold interface; and
a volatile liquid disposed at the hot interface, the volatile liquid configured to remove the heat from the conductive plate and phase-change to a vapor.

22. The portable cooling unit of claim 21, wherein:
the cold interface is configured to receive and condense the vapor to the volatile liquid; and
the conductive tunnel is configured to transport the condensed volatile liquid from the cold interface to the hot interface.

23. The portable cooling unit of claim 21, wherein the conductive tunnel comprises an interior surface configured to transport of the condensed volatile liquid via capillary action.

24. A portable cooling unit, comprising:
a conductive plate having a first side and a second side, the first side of the conductive plate configured to interface with a palm of a user and configured to be secured with the palm of the user via at least one connecting strap;
a heat sink having an upper side, a lower side, and a body thermally connecting the upper side and the lower side, the upper side of the heat sink positioned facing the second side of the conductive plate, the heat sink comprising a plurality of fins disposed on the body, the plurality of fins being configured to increase a surface area of the heat sink for heat dissipation;
a fan having an air outlet side and an air inlet side, the fan connected to the heat sink such that the air outlet side faces the lower side of the heat sink;
a power source connected to the fan via at least one connecting cable and configured to be secured to a wrist of the user via at least one wrist strap; and
one or more heat pipes thermally connecting the conductive plate to the heat sink.

25. A portable cooling unit, comprising:
a conductive plate having a first side and a second side;
a heat sink having an upper side, a lower side, and a body thermally connecting the upper side and the lower side, the upper side of the heat sink positioned facing the second side of the conductive plate, the heat sink comprising a plurality of fins disposed on the body, the plurality of fins being configured to increase a surface area of the heat sink for heat dissipation;
a power source; and
a Peltier module electrically connected to the power source via at least one connecting cable, the Peltier Module comprising:
a heat absorbing end thermally connected to the second side of the conductive plate and configured to absorb heat from the conductive plate;
a heat releasing end thermally connected to the upper side of the heat sink and configured release the absorbed heat from the conductive plate into the heat sink; and
a plurality of semiconductor elements electrically connected to the power source, the plurality of semiconductor elements thermally connecting the heat absorbing end and the heat releasing end.

26. The portable cooling unit according to claim 25, further comprising a fan having an air outlet side and an air inlet side, the fan connected to the heat sink such that the air outlet side is positioned facing the lower side of the heat sink, and the power source connected to the fan via at least one connecting cable.

27. The portable cooling unit according claim 25 or claim 26, wherein the first side of the conductive plate is configured to interface with a palm of a user.

28. The portable cooling unit according to claim 27, wherein the first side of the conductive plate is configured to be secured with the palm of the user via at least one connecting strap.

29. The portable cooling unit according to any one of claims 25-28, wherein the power source is a battery configured to be secured to a wrist of the user via at least one wrist strap.

30. The portable cooling unit according to any one of claims 25-28, wherein the power source is positioned on a part of the user's body other than the wrist of the user.

31. The portable cooling unit according to any one of claims 25-30, further comprising at least one temperature probe disposed on the first side of the conductive plate, the at least one temperature probe configured to measure a surface temperature of the first side of the conductive plate.

32. The portable cooling unit according to claim 31, further comprising a user interface configured for interaction with the user, the user interface comprising:
a graphic user interface (GUI) disposed on the conductive plate;
a processor coupled with the GUI;
a memory coupled with and readable by the processor and storing therein a set of instructions which, when executed by the processor, causes the processor to perform operations comprising:
(a) receiving a user input via the GUI, the user input comprising a range of target surface temperatures of the first side of the conductive plate for maintaining a target core body temperature of the user;
(b) receiving the surface temperature of the first side of the conductive plate from the at least one temperature probe;
(c) determining if the surface temperature of the first side of the conductive plate is within the range of target surface temperature; and
if the surface temperature of the first side of the conductive plate is greater than the range of target surface temperature,
(d) activating the Peltier module or the fan, or both, for a period of time;
(e) repeating steps (b)-(d) until the surface temperature of the first side of the conductive plate is within the range of target surface temperature; and
(f) deactivating the fan when the surface temperature of the first side of the conductive plate is within the range of target surface temperature.

33. The portable cooling unit according to any one of claims 25-32, wherein heat dissipation from the heat sink occurs via at least one of radiation, conduction, or convection.

34. The portable cooling unit according to any one of claims 25-33, wherein the conductive plate further comprises an insulation layer disposed on the first side of the conductive plate, the insulative layer configured to prevent loss of heat from the first side of the conductive plate.

35. A portable cooling unit, comprising:
a conductive plate having a first side and a second side;
a heat sink having an upper side, a lower side, and a body thermally connecting the upper side and the lower side, the upper side of the heat sink positioned to facing the second side of the conductive plate, the heat sink comprising a plurality of fins disposed on the body, the plurality of fins being configured to increase a surface area of the heat sink for heat dissipation;
a fan having an air outlet side and an air inlet side, the fan connected to the heat sink such that the air outlet side is positioned faces the lower side of the heat sink,
a power source connected to the fan via at least one connecting cable;
one or more heat pipes thermally connecting the conductive plate to the heat sink; and
a Peltier module electrically connected to the power source via at least one connecting cable, the Peltier Module comprising:
a heat absorbing end thermally connected to the second side of the conductive plate and configured to absorb heat from the conductive plate;
a heat releasing end thermally connected to the upper side of the heat sink and configured release the absorbed heat from the conductive plate into the heat sink; and
a plurality of semiconductor elements electrically connected to the power source, the plurality of semiconductor elements thermally connecting the heat absorbing end and the heat releasing end.

36. The portable cooling unit of claim 35, wherein:
the heat absorbing end of the Peltier module is thermally connected to the one or more heat pipes and configured to absorb heat from the one or more heat pipes; and
the heat releasing end of the Peltier module is configured to release the absorbed hear from the one or more heat pipes into the heat sink.

37. A portable cooling unit, comprising:
a conductive plate having a first side and a second side;
a heat sink having an upper side, a lower side, and a body thermally connecting the upper side and the lower side, the upper side of the heat sink positioned facing the second side of the conductive plate, the heat sink comprising a plurality of fins disposed on the body, the plurality of fins being configured to increase a surface area of the heat sink for heat dissipation;
a fan having an air outlet side and an air inlet side, wherein the fan is connected to the heat sink such that the air outlet side is positioned to face the lower side of the heat sink,
a power source connected to the fan via at least one connecting cable;
one or more heat pipes; and
a Peltier module electrically connected to the power source via at least one connecting cable, the Peltier Module comprising:
a heat absorbing end thermally connected to the one or more heat pipes and configured to absorb heat from the one or more heat pipes;
a heat releasing end thermally connected to the upper side of the heat sink and configured release the absorbed heat from the one or more heat pipes into the heat sink; and
a plurality of semiconductor elements electrically connected to the power source, the plurality of semiconductor elements thermally connecting the heat absorbing end and the heat releasing end.

38. The portable cooling unit according to claim 37, wherein the first side of the conductive plate is configured to interface with a palm of a user.

39. The portable cooling unit according to claim 38, wherein the first side of the conductive plate is configured to be secured with the palm of the user via at least one connecting strap.

40. The portable cooling unit according to any one of claims 37-39, wherein the power source is a battery configured to be secured to a wrist of the user via at least one wrist strap.

41. The portable cooling unit according to any one of claims 37-39, wherein the power source is a battery positioned on a part of the user's body other than the wrist of the user.

42. The portable cooling unit according to any one of claims 37-41, further comprising at least one temperature probe disposed on the first side of the conductive plate, the at least one temperature probe configured to measure a surface temperature of the first side of the conductive plate.

43. The portable cooling unit according to claim 42, further comprising a user interface configured for interaction with the user, the user interface comprising:
a graphic user interface (GUI) disposed on the conductive plate;
a processor coupled with the GUI;
a memory coupled with and readable by the processor and storing therein a set of instructions which, when executed by the processor, causes the processor to perform operations comprising:
(a) receiving a user input via the GUI, the user input comprising a range of target surface temperatures of the first side of the conductive plate for maintaining a target core body temperature of the user;
(b) receiving the surface temperature of the first side of the conductive plate from the at least one temperature probe;
(c) determining if the surface temperature of the first side of the conductive plate is within the range of target surface temperature; and
if the surface temperature of the first side of the conductive plate is greater than the range of target surface temperature,
(d) activating the Peltier module or the fan, or both, for a period of time;
(e) repeating steps (b)-(d) until the surface temperature of the first side of the conductive plate is within the range of target surface temperature; and
(f) deactivating the pump and the fan when the surface temperature of the first side of the conductive plate is within the range of target surface temperature.

44. The portable cooling unit according to any one of claims 37-43, wherein heat dissipation from the heat sink occurs via at least one of radiation, conduction, or convection.

45. The portable cooling unit according to any one of claims 37-44, wherein the conductive plate further comprises an insulation layer disposed on the first side of the conductive plate, the insulative layer configured to prevent loss of heat from the first side of the conductive plate.

46. The portable cooling unit according to any one of claims 37-45, wherein the one or more heat pipes are made from a thermally conductive material.

47. The portable cooling unit according to any one of claims 37-46, wherein the one or more heat pipes comprise between two and thirty heat pipes.

48. The portable cooling unit according to any one of claims 37-47, wherein the one or more heat pipes comprise six heat pipes.

49. The portable cooling unit according to any one of claims 37-48, wherein each of the one or more heat pipes comprises:
a hot interface thermally connected to the conductive plate;
a cold interface thermally connected to the Peltier module;
a conductive tunnel connecting the hot interface and the cold interface; and
a volatile liquid disposed at the hot interface, the volatile liquid configured to remove the heat from the conductive plate and phase-change to a vapor.

50. The portable cooling unit of claim 49, wherein:
the cold interface is configured to receive and condense the vapor to the volatile liquid; and
the conductive tunnel is configured to transport the condensed volatile liquid from the cold interface to the hot interface.

51. The portable cooling unit of claim 50, wherein the conductive tunnel comprises an interior surface configured to transport of the condensed volatile liquid via capillary action.

52. A portable cooling unit, comprising:
a conductive plate having a first side and a second side;
a heat sink having an upper side, a lower side, and a body thermally connecting the upper side and the lower side, the upper side of the heat sink positioned facing the second side of the conductive plate, the heat sink comprising a plurality of fins disposed on the body, the plurality of fins being configured to increase a surface area of the heat sink for heat dissipation;
a fan connected to the power source via at least one connecting cable, the fan having an air outlet side and an air inlet side, the fan connected to the heat sink such that the air outlet side is positioned facing the lower side of the heat sink;
a power source connected to the fan via at least one connecting;
one or more heat pipes thermally connecting the conductive plate to the heat sink;
a liquid cooling system comprising:
an internal fluid chamber thermally connected to the second side of the conductive plate, the internal fluid chamber comprising conductive tubing; and
a pump electrically connected to the power source via at least one connecting cable and fluidically connected to the internal fluid chamber via at least one fluid port, the pump configured to circulate a coolant through the conductive tubing of the internal fluid chamber, the coolant configured to remove heat from the conductive plate via convection;
and
a Peltier module electrically connected to the power source via at least one connecting cable, the Peltier Module comprising:
a heat absorbing end thermally connected to the one or more heat pipes and configured to absorb heat from the one or more heat pipes;
a heat releasing end thermally connected to the upper side of the heat sink and configured release the absorbed heat from the one or more heat pipes into the heat sink; and
a plurality of semiconductor elements electrically connected to the power source, the plurality of semiconductor elements thermally connecting the heat absorbing end and the heat releasing end.

53. The portable cooling unit according to claim 52, wherein the first side of the conductive plate is configured to interface with a palm of a user.

54. The portable cooling unit according to claim 53, wherein the first side of the conductive plate is configured to be secured with the palm of the user via at least one connecting strap.

55. The portable cooling unit according to claim 53, wherein the power source is configured to be secured to a wrist of the user via at least one wrist strap.
